(19) United States
(12) Patent Application Publication
(10) Pub. No.: US 2024/0071625 A1
(43) Pub. Date: Feb. 29, 2024

(54) NAFLD IDENTIFICATION AND PREDICTION SYSTEMS AND METHODS

(71) Applicant: CEDARS-SINAI MEDICAL CENTER, Los Angeles, CA (US)

(72) Inventors: Mazen Noureddin, Houston, TX (US); Devon Chang, Los Angeles, CA (US)

(21) Appl. No.: 18/457,922

(22) Filed: Aug. 29, 2023

Related U.S. Application Data
(60) Provisional application No. 63/402,039, filed on Aug. 29, 2022.

(51) Int. Cl. G16H 50/30; G16H 50/20; G16H 50/70

(52) U.S. Cl. CPC G16H 50/30; G16H 50/20; G16H 50/70

(57) ABSTRACT

A system for identifying and predicting the presence of non-alcoholic fatty liver disease (NAFLD). The system can receive historical biological data associated with multiple individuals. The historical biological data can include biological characteristics. The system can use the historical biological data to generate and train machine learning models to predict the presence of NAFLD in a liver. The machine learning models can generate a prediction related to NAFLD or can generate a formula, which can be used to generate the prediction. The system can receive biological data associated with an individual. The system can apply the machine learning models or the formulas to the biological data to generate a liver score for the individual. The liver score can indicate a predicted liver fibrosis stage and NAFLD activity score (NAS).

### CROSS-REFERENCE TO RELATED APPLICATIONS

This application claims the benefit of, and priority to, U.S. Provisional Patent Application No. 63/402,039, filed on Aug. 29, 2022 and entitled “Systems and Methods for Identifying Stages of NAFLD and NAFLD-Related Cirrhosis,” the entirety of which is incorporated by reference as if fully set forth herein.

### TECHNICAL FIELD

The present disclosure relates identifying and predicting liver health using machine learning models.

### BACKGROUND

Non-alcoholic fatty liver disease (NAFLD) is one of the most common liver diseases. NAFLD affects over 25% of the general population and carries a huge cost in our healthcare system. Over time, NAFLD can progress to more severe diseases such as non-alcoholic steatohepatitis (NASH) and cirrhosis. Patients with NASH are at increased risk for cirrhosis and death, but may also benefit from pharmacotherapies. NASH is histologically assessed by the Brunt criteria and numerically upon trial entry using the NAFLD disease activity score (NAS).

Current methods for identifying NAFLD and/or NASH, including FibroScan, Fibrosis-4 (FIB-4), magnetic resonance imaging (MRI) elastography, NAFLD fibrosis score (NFS), and liver biopsy, can be invasive, risky, and/or expensive. For example, liver biopsy is the current reference standard for identifying liver fibrosis. However, liver biopsy is an invasive procedure with risk of sampling error, high cost, and risks of complications. FibroScan and MRIs are costly and/or not widely available in gastrointestinal (GI) or primary care clinics. FIB-4 and NFS can only identify those with fibrosis stage F3 or higher, and both tests have a large indeterminate zone. Therefore, there is a long-felt but unresolved need to predicting and identifying NAFLD and other liver diseases in a low-cost, low-risk, and simple manner.

### SUMMARY

Briefly described, and according to one embodiment, aspects of the present disclosure generally relate to identifying and predicting the presence of non-alcoholic fatty liver disease (NAFLD), liver fibrosis, non-alcoholic steatohepatitis (NASH), cirrhosis, and liver failure in a liver of an individual. The system can receive historical biological data, including various biological characteristics related to NAFLD. The system can select biological features from among the historical biological data to generate and train machine learning models. The machine learning models can be trained to predict NAFLD in a liver. The system can receive biological data associated with an individual and apply the machine learning models to the biological data to generate a prediction. The prediction can include an indication of a liver fibrosis stage and NAFLD activity score (NAS).

According to aspects of the present disclosure, the system can include at least one electronic interface, at least one memory device, and at least one processing device. The at least one electronic interface is configured to receive data associated with one or more biological characteristics of the individual. The at least one memory device is configured to store the data associated with the one or more biological characteristics of the individual. The at least one processing device is configured to implement one or more machine learning models. The one or more machine learning models are configured to receive data associated with at least one of the one or more biological characteristics of the individual and predict the presence of a liver disease in the liver of the individual.

For the purpose of promoting an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

Whether a term is capitalized is not considered definitive or limiting of the meaning of a term. As used in this document, a capitalized term shall have the same meaning as an uncapitalized term, unless the context of the usage specifically indicates that a more restrictive meaning for the capitalized term is intended. However, the capitalization or lack thereof within the remainder of this document is not intended to be necessarily limiting unless the context clearly indicates that such limitation is intended.

The foregoing and additional aspects and implementations of the present disclosure will be apparent to those of ordinary skill in the art in view of the detailed description of various embodiments and/or implementations, which is made with reference to the drawings, a brief description of which is provided next.

### BRIEF DESCRIPTION OF THE DRAWINGS

The foregoing and other advantages of the present disclosure will become apparent upon reading the following detailed description and upon reference to the drawings. The accompanying drawings illustrate one or more embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1 illustrates an exemplary high-level overview for the disclosed system, according to aspects of the present disclosure.

FIG. 2 illustrates an exemplary networked environment for the disclosed system, according to aspects of the present disclosure.

FIG. 3 shows illustrates an exemplary overall process for the disclosed system, according to aspects of the present disclosure.

FIG. 4 illustrates an exemplary table comparing the performance of various machine learning models, according to aspects of the present disclosure.

FIG. 5 illustrates an exemplary table comparing the performance of various machine learning models against other diagnostic methods, according to aspects of the present disclosure.

FIG. 6 illustrates an exemplary table comparing the performance of various machine learning models against other diagnostic methods, according to aspects of the present disclosure.

FIG. 7 illustrates an exemplary table comparing the performance of various machine learning models against other diagnostic methods, according to aspects of the present disclosure.

FIG. 8 shows plots of the true positive rate versus the false positive rate for various machine learning models against other diagnostic methods, according to aspects of the present disclosure.

FIG. 9 illustrates an exemplary computing system for the disclosed system, according to aspects of the present disclosure.

While the present disclosure is susceptible to various modifications and alternative forms, specific implementations and embodiments have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

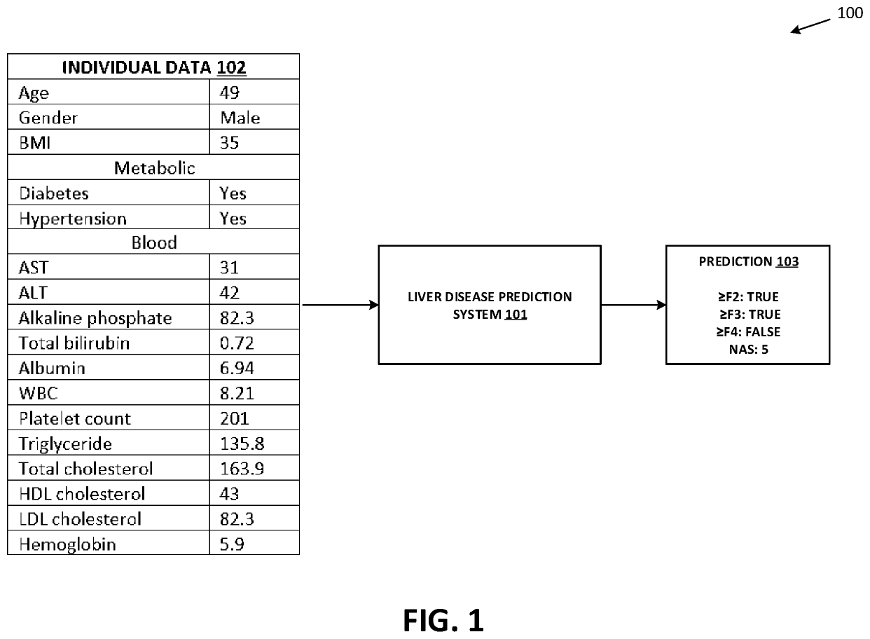

Referring now to the figures, for the purposes of example and explanation of the fundamental processes and components of the disclosed systems and processes, reference is made to FIG. 1, which illustrates an exemplary, high-level overview 100 of liver disease prediction system 101. As will be understood and appreciated, the exemplary system 101 shown in FIG. 1 represents merely one approach or embodiment of the present system, and other aspects are used according to various embodiments of the present system.

As will be understood by one having ordinary skill in the art, the steps and processes shown in FIG. 1 (and those of all other flowcharts and sequence diagrams shown and described herein) may operate concurrently and continuously, are generally asynchronous and independent, and are not necessarily performed in the order shown. The liver disease prediction system 101 can predict a liver disease, including liver fibrosis, NAFLD, NASH, cirrhosis, and liver failure, in an individual. The liver disease prediction system 101 can apply machine learning models to data describing biological characteristics associated with an individual to generate one or more predictions. The liver disease prediction system 101 can receive individual data 102, which can include biological characteristics, including but not limited to gender, age, body mass index (BMI), alkaline phosphatase level, total bilirubin level, alanine transaminase (ALT) level, aspartate aminotransferase (AST) level, albumin level, white blood count (WBC), platelet count, hemoglobin A1C level, total cholesterol level, low-density lipoprotein (LDL) cholesterol level, high-density lipoprotein (HDL) level, triglycerides level, type 2 diabetes status, and hypertension status. The biological characteristics can be determined by non-invasive measures (e.g., blood draw) that can be performed without specialized equipment and by any medical professional. The foregoing biological characteristics can be determined by a review of an individual's medical history and may not require any additional testing or procedures.

The individual data 102 can include biological characteristics closely related to the pathophysiology and disease progression of NAFLD. Biological characteristics including gender, age, BMI, hemoglobin A1C, total cholesterol, LDL, HDL, triglycerides, type 2 diabetes status, and hypertension can show strong connections to metabolic syndrome and NAFLD. Biological characteristics, including platelet count, total bilirubin, ALT, AST, and albumin can indicate disease severity (e.g., NAFLD severity). Biological characteristics, including white blood cell count, can be related to innate immune activation, which can identify hepatic inflammation in NAFLD and NASH. Biological characteristics, including alkaline phosphate levels, can be related to bile acid regulation pathways, for example ligands of the farnsoid-X-receptor (FXR) and G protein bile acid receptor (GPBAR)1, which can show efficacy in protecting against NAFLD progression and reversing inflammation and fibrosis.

After receiving the individual data 102, the liver disease prediction system 101 can apply machine learning models to the individual data 102 to generate the prediction 103. The machine learning models can be generated by the liver disease prediction system 101 based on historical biological data. The machine learning models can generate, produce, or derive a resulting formula to generate the prediction 103 or the machine learning models can include the resulting formula. The prediction 103 can include an indication of the fibrosis stage of the individual's liver. In some embodiments, the fibrosis stage can be F0 (e.g., no fibrosis), F1 (e.g., minimal fibrosis), F2 (e.g., significant fibrosis), F3 (e.g., advanced fibrosis), and F4 (e.g., cirrhosis). The prediction 103 can include an NAFLD activity score (NAS). In some embodiments, the NAS can be a numerical value that is greater than or equal to 0, and less than or equal to 8. A higher NAS can indicate that NASH is likely present in the individual's liver. The NAS can have different components that can indicate different measures of the liver. For example, the NAS can include a first component indicative of steatosis of the liver, a second component indicative of lobular inflammation in the liver, and a third component indicative of hepatocyte ballooning in the liver. The prediction 103 can include an indication of the fibrosis stage and the NAS. For example, the prediction 103 can include an indication of whether non-alcoholic steatohepatitis (NASH) is present in the individual's liver. NASH is generally a severe form of NAFLD, and can be indicated by a fibrosis stage of greater than or equal to F2, and a NAS of greater than or equal to 4.

The system 101 can apply machine learning models to the individual data 102 to determine the different fibrosis stages and the different NAS scores. For example, the one or more machine learning models can include at least a first machine learning model, a second machine learning model, and a third machine learning model. The first machine learning model can be trained to predict if the fibrosis stage of the liver is greater than or equal to F2. The second machine learning model can be trained to predict if the fibrosis stage of the liver is greater than or equal to F3. The third machine learning model can be trained to predict if the fibrosis stage of the liver is greater than or equal to F4. In this example, the predictions of each machine learning model can be combined to determine the exact fibrosis stage of the liver. For example, the machine learning models can be trained to predict the fibrosis stage of the liver (e.g., F2, F3, F4), instead of a prediction that the fibrosis stage is at least equal to a given stage. For example, the machine learning models can predict the NAS, where each machine learning model has been trained to output an indication of whether the NAS is greater than or equal to a given NAS (e.g., 0, 1, 2). For example, a machine learning model can be trained to predict the NAS (e.g, 0, 1, 2, etc.), instead of a determination that the NAS is at least equal to given value.

In some embodiments, one or more machine learning models can be trained to predict a single indication of the fibrosis stage and the NAS. The machine learning models can be trained to predict a single indication of whether the fibrosis stage is at least equal to a given value and whether the NAS is at least equal to a given value. For example, a first machine learning model can predict if the fibrosis stage is greater than or equal to F2 and the NAS is greater than or equal to 1. A second machine learning model can predict if the fibrosis stage is greater than or equal to F3 and the NAS is greater than or equal to 5. A third machine learning model can predict if the fibrosis stage is greater than or equal to F4 and the NAS is greater than or equal to 7.

As noted herein, the presence of NASH can be indicated by a fibrosis stage of at least F2, and a NAS of at least 4. In some embodiments, the one or more machine learning models can include at least three machine learning models. A first machine learning model can predict if the fibrosis stage is greater than or equal to F2 and the NAS is greater than or 4. A second machine learning model can predict if the fibrosis stage is greater than or equal to F3 and the NAS is greater than or 4. A third machine learning model can predict if the fibrosis stage is greater than or equal to F4 and the NAS is greater than or 4. For example, a machine learning model can predict if the fibrosis stage is greater than or equal to F2 and the NAS is greater than or 4.

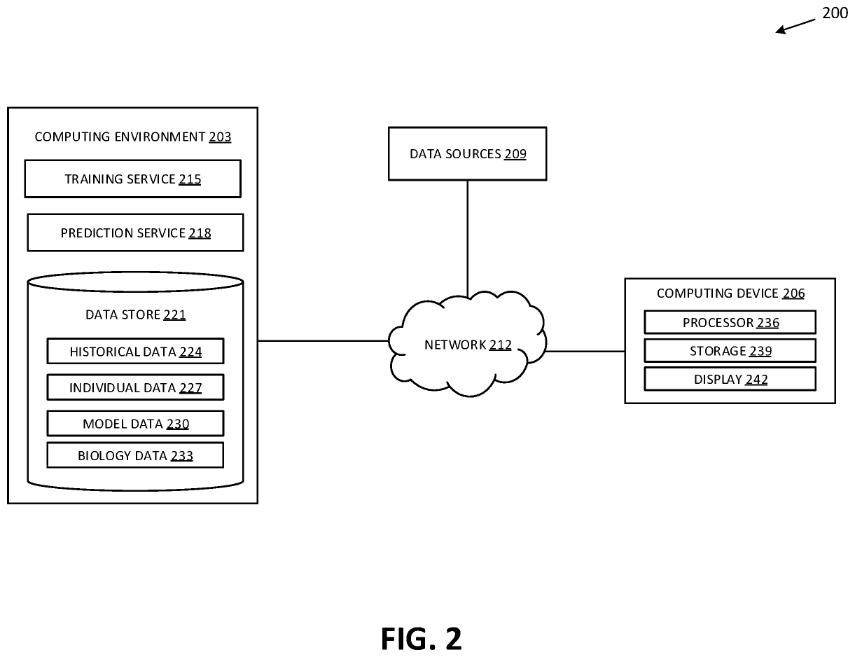

Referring now to FIG. 2, shown is an exemplary networked environment 200 for the liver disease prediction system according to various embodiments of the present disclosure. As will be understood and appreciated, the exemplary networked environment 200 shown in FIG. 2 represents merely one approach or embodiment of the present system, and other aspects are used according to various embodiments of the present system. Exemplary networked environment 200 can include, but is not limited to, a computing environment 203 connected to one or more computing devices 206 and data sources 209 over a network 212.

The elements of the computing environment 203 can be provided via one or more computing devices that may be arranged, for example, in one or more server banks or computer banks or other arrangements. Such computing devices can be located in a single installation or may be distributed among many different geographical locations. For example, the computing environment 203 can include one or more computing devices that together may include a hosted computing resource, a grid computing resource, or any other distributed computing arrangement. In some cases, the computing environment 203 can correspond to an elastic computing resource where the allotted capacity of processing, network, storage, or other computing-related resources may vary over time. Regardless, the computing environment 203 can include one or more processors and memory having instructions stored thereon that, when executed by the one or more processors, cause the computing environment 203 to perform one, some, or all of the actions, methods, steps, or functionalities provided herein.

The computing environment 203 can include a training service 215, a prediction service 218, and a data store 221. The training service 215 and the prediction service 218 can correspond to one or more software executables that can be executed by the computing environment 203 to perform the functionality described herein. While the training service 215 and the prediction service 218 are described as different services, it can be appreciated that the functionality of these services can be implemented in one or more different services executed in the computing environment 203. Various data can be stored in the data store 221, including but not limited to, historical data 224, the individual data 227, the model data 230, and the biology data 233.

The training service 215 can generate and train multiple machine learning models to predict a liver disease, including liver fibrosis, NAFLD, NASH, cirrhosis, and liver failure. The training service 215 can receive historical biological data as the historical data 224. The historical biological data can include biological characteristics for an associated individual including but not limited to gender, age, body mass index (BMI), alkaline phosphatase level, total bilirubin level, alanine transaminase (ALT) level, aspartate aminotransferase (AST) level, albumin level, white blood count (WBC), platelet count, hemoglobin A1C level, total cholesterol level, low-density lipoprotein (LDL) cholesterol level, high-density lipoprotein (HDL) level, triglycerides level, type 2 diabetes status, and hypertension status. The training service 215 can receive a diagnosis and/or non-diagnosis for the individuals associated with the historical biological data. The diagnosis and/or non-diagnosis can indicate if the individual has been diagnosed with a liver disease. The training service 215 can use the biological characteristics as input variables and the diagnosis and/or non-diagnosis as output variables to generate and train the machine learning models.

The training service 215 can generate and train classification models, including but not limited to logistic regression models, random forest models, and artificial neural networks. The machine learning models can predict a liver disease as a binary output (e.g., true, false). For example, the machine learning model can predict whether or not an individual has F2 or greater liver fibrosis. The machine learning models can be saved as the model data 230 in the data store.

The prediction service 218 can apply the machine learning models to biological data for an individual to predict if the individual has a liver disease. The prediction service 218 can receive biological data for the individual. The prediction service 218 can receive the biological data as inputs to an electronic display on the display 242. The prediction service 218 can apply the machine learning models to the biological data for the individuals to generate a prediction. The prediction can be indicated by a liver score. Each machine learning model can generate a liver score and the liver scores individually generated by the machine learning models can be combined to create an overall liver score. The liver score can include a fibrosis stage prediction and a NAS prediction. For example, the liver score can indicate whether the fibrosis stage is greater than or equal to F2 and a NAS is greater than or equal to 4. The prediction service 218 can use the liver score to determine a fibrosis stage and diagnose a liver disease.

According to various embodiments, the computing device 206 can include any device capable of accessing network 212 including, but not limited to, a computer, smartphone, tablets, or other device. The computing device 206 can include a processor 236 and storage 239. The computing device 206 can include a display 242 on which various user interfaces can be rendered to allow users to configure, monitor, control, and command various functions of networked environment 200. In various embodiments, computing device 206 can include multiple computing devices. Regardless, the computing device 206 can include one or more processors and memory having instructions stored thereon that, when executed by the one or more processors, cause the computing device 206 to perform one, some, or all of the actions, methods, steps, or functionalities provided herein.

The network 212 includes, for example, the Internet, intranets, extranets, wide area networks (WANs), local area networks (LANs), wired networks, wireless networks, or other suitable networks, etc., or any combination of two or more such networks.

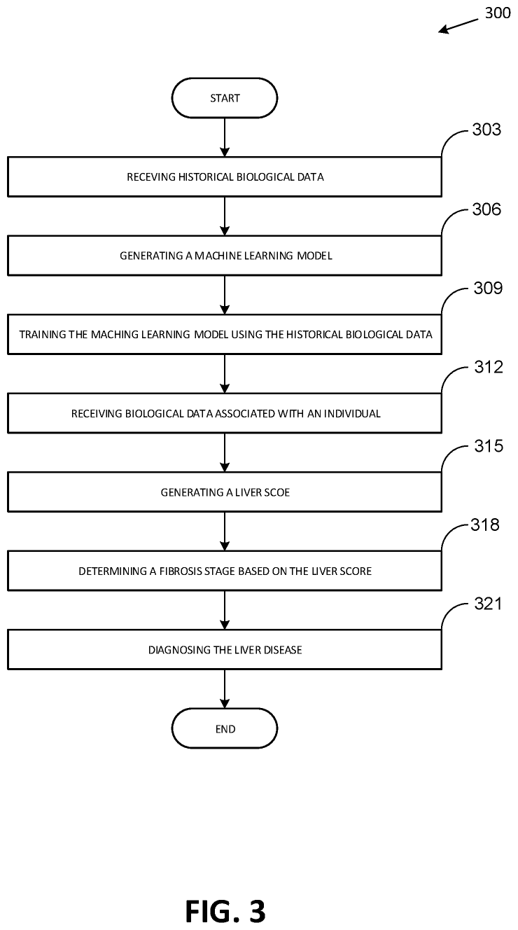

Referring now to FIG. 3, shown is an exemplary, high-level overview process 300 for according to various embodiments of the present disclosure. As will be understood by one having ordinary skill in the art, the steps and processes shown in FIG. 3 may operate concurrently and continuously, are generally asynchronous and independent, can be performed in part or in whole by a combination of one or more of the computing environment 203 and computing devices 206, and are not necessarily performed in the order shown and various steps can be executed linearly or in parallel. Process 300 can be performed entirely, partially, or in coordination with the training service 215 and prediction service 218.

At step 303, the process 300 can include receiving historical biological data. The training service 215 can receive the historical biological data from the computing device 206, the data sources 209, and/or the historical data 224. The received historical biological data can be stored in the data store 221 as the historical data 224. The historical biological data can include multiple sets of historical biological data and each set of historical biological data can be associated with an individual. Each set of historical biological data can include at least one diagnosis or non-diagnosis of a liver disease. For example, each set of historical biological data can include an indication of the individual associated with the set of historical biological data has been diagnosed or not diagnosed with a liver disease. For example, if the set of historical biological data includes a diagnosis for a liver disease, the diagnosis can indicate that the associated individual has been diagnosed with that liver disease. For example, if the set of historical biological data includes a non-diagnosis for a liver disease, the non-diagnosis can indicate that the associated individual has not been diagnosed with that liver disease. The diagnosed and/or non-diagnosed liver diseases can include non-alcoholic fatty liver disease (NAFLD), non-alcoholic steatohepatitis (NASH), cirrhosis, and liver failure. The diagnosis and/or non-diagnosis can include a fibrosis stage. A fibrosis stage can indicate the severity of fibrosis in the individual's liver. Fibrosis stages can range from F0 to F4, specifically F0 (e.g., no fibrosis), F1 (e.g., minimal fibrosis), F2 (e.g., significant fibrosis), F3 (e.g., advanced fibrosis), and F4 (e.g., cirrhosis). The diagnosis and/or non-diagnosis can include a NAFLD activity score (NAS), which can indicate the severity of NASH or cirrhosis. NAS can range from 0 to 8, with a higher values (e.g., higher in the 0 to 8 range) indicating more severe NASH and/or cirrhosis.

The historical biological data can include biological characteristics for the associated individual, including but not limited to gender, age, body mass index (BMI), alkaline phosphatase level, total bilirubin level, alanine transaminase (ALT) level, aspartate aminotransferase (AST) level, albumin level, white blood count (WBC), platelet count, hemoglobin A1C level, total cholesterol level, low-density lipoprotein (LDL) cholesterol level, high-density lipoprotein (HDL) level, triglycerides level, type 2 diabetes status, and hypertension status. Each set of historical biological data can include a combination of any of the foregoing biological characteristics.

At step 306, the process 300 can include generating one or more machine learning models. The training service 215 can generate the machine learning model. The machine learning models can be stored in the data store 221 as the model data 230. The machine learning model can predict a liver disease, including liver fibrosis, NAFLD, NASH, cirrhosis, and liver failure. The historical biological data received at step 303 can be used as the inputs and outputs for generating the machine learning models. The biological characteristics can be used as the inputs and the diagnosis and/or non-diagnosis can be used at the outputs. As an example, the training service 215 can train the machine learning model to identify a particular diagnosis by analyzing the historical biological data including biological data corresponding to confirmed diagnoses for a particular disease (e.g., has F2 or greater liver fibrosis) and biological data corresponding to confirmed non-diagnoses for the particular disease (e.g., F2 or greater liver fibrosis) to create a model predictive of whether a test individual has the particular disease (e.g., F2 or greater liver fibrosis). For example, the biological characteristics included in the sets of biological data can be selected as features for the machine learning models. Selecting features from the biological characteristics can eliminate any irrelevant, inaccurate, or duplicate data included in the historical biological data. In some embodiments, the output of the machine learning models can be a prediction related to liver disease. In some embodiments, the output of the machine learning models can be one or more resulting formula. The system can use the resulting formula to generate a prediction related to liver disease. The resulting formula can be included in the machine learning models. The prediction service 218 can apply the resulting one or more formulas to generate predictive scores for one or more diseases (e.g., predictive of having F2 or greater liver fibrosis, or predictive of having F3 or greater liver fibrosis). The predictions service can determine whether results of one or more formulas meets or exceeds one or more thresholds. The prediction service 218 can determine a recommended diagnosis based on whether the results of the one or more formulas meets the one or more thresholds. As an example, the prediction service 218 can recommend a diagnosis of F3 or greater liver fibrosis in response to a formula corresponding to this diagnosis producing a result that meets or exceeds a predefined threshold.

The machine learning models can be classification models that can predict whether an individual has liver fibrosis. The prediction can include a binary prediction (e.g., true, false). For example, the machine learning model can predict whether or not an individual has F2 or greater liver fibrosis. For example, the machine learning model can predict whether or not an individual has a NAS of 4 or greater. The machine learning models can include a logistic regression model. The logistic regression model can model the outputs as a sigmoid function of the inputs using logit scaling. The logistic regression model can assume that there is little or no correlation between the input variables. For example, if the logistic regression model received the gender and AST level for an individual, the logistic regression model can assume that there is little or no correlation between the gender and AST level.

The machine learning models can include a random forest model. The random forest model can include multiple decision tree models. The decision tree models can be generated using random subsets of the historical biological data or the biological features. To generate the decision tree models, the random subsets can be recursively split based on a biological feature or characteristic. The subset can continue to split until splitting no longer improves the prediction of the decision tree model. The decision tree models can have a specified maximum depth or no maximum depth (e.g., a maximum tree depth or no maximum tree depth). Each decision tree model can generate a prediction. The random forest model can combine the predictions of each decision tree model and output the highest ranking prediction (e.g., the most popular prediction among the decision trees, the prediction with the highest confidence). The random forest can include any number of decision tree models (e.g., 3 decision trees, 10 decision trees, 30 decision trees, 50 decision trees).

The machine learning models can include an artificial neural network. The artificial neural network can include an input layer for receiving input variables, multiple hidden layers for processing the input variables, and an output layer for producing or generating output variables. The artificial neural network can have any number of hidden layers. For example, the artificial neural network can have two or more hidden layers.

At step 309, the process 300 can include training the machine learning models using the historical biological data. The training service 215 can train the machine learning models. The machine learning models can be trained using the sets of historical biological data, including the biological characteristics and biological features. A portion of the historical biological data can be excluded from training for testing and evaluating the machine learning models. For example, 20% or more of the historical biological data can be excluded from the training set (e.g., the portion of the historical biological data used to train the machine learning models). The excluded portion of the historical biological data can be used as a testing or evaluation set. The machine learning models can be evaluated or tested using the testing set. For example, the machine learning models can use the historical biological data to confirm that the machine learning models are accurately predicting whether an individual has disease, such as liver fibrosis.

At step 312, the process 300 can include receiving biological data associated with an individual. The prediction service 218 can receive the biological data associated with an individual. The received biological data can be stored in the data store as the individual data 227. The biological data can include biological characteristics for the associated individual, including but not limited to gender, age, body mass index (BMI), alkaline phosphatase level, total bilirubin level, alanine transaminase (ALT) level, aspartate aminotransferase (AST) level, albumin level, white blood count (WBC), platelet count, hemoglobin A1C level, total cholesterol level, low-density lipoprotein (LDL) cholesterol level, high-density lipoprotein (HDL) level, triglycerides level, type 2 diabetes status, and hypertension status. The biological data can be received via an electronic user interface. For example, the biological data can be received as inputs from a user.

At step 315, the process 300 can include generating a liver score. The prediction service 218 can generate a liver score. The liver score can be generated by applying the machine learning models or the resulting formula generated by the machine learning models to the biological data received at step 312. The liver score can indicate the liver disease prediction generated by the machine learning models for the individual associated with the biological data received at step 312. Each machine learning model can generate a liver score and the liver scores individually generated by the machine learning models can be combined to create an overall liver score. The liver score can include a fibrosis stage prediction and a NAS prediction. The liver score can include multiple scores, each indicating a fibrosis stage prediction. For example, a first score can predict if the individual has fibrosis stage of F2 or greater, a second score can predict if the individual has fibrosis stage of F3 or greater, and a third score can predict if the individual has fibrosis stage of F4. For example, the first score can predict that the individual has fibrosis stage of F2 or greater, the second score can predict that the individual has fibrosis of F3 or greater, and the third score can predict that the individual does not have fibrosis stage of F4. In this example, the liver score can indicate that the individual has F3 stage fibrosis, but not F4 stage fibrosis.

The liver score can include multiple scores, each indicating a NAS prediction. For example, the liver score can include multiple scores, each score indicating whether the NAS is greater than or equal to a value (e.g., a first score indicating whether NAS is greater than or equal to 1, a second score indicating whether NAS is greater than or equal to 2). The liver score can include 8 scores, each predictive of NAS values (e.g., NAS can be a value between 0 and 8). The liver score could include a prediction of NAS. For example, rather than predicting whether the NAS is greater than or equal to a value, the liver score can predict the NAS value. The liver score can indicate multiple predictions. For example, the liver score can indicate whether the fibrosis stage is greater than or equal to F2 and a NAS is greater than or equal to 4. For example, the liver score can indicate whether the fibrosis stage is greater than or equal to F3 and a NAS is greater than or equal to 5.

The liver score can be generated by applying Youden's Index to the machine learning models. Youden's Index can be used to determine the binary prediction made by the machine learning models. As will be understood and appreciated, to make a binary prediction, a machine learning models can generate a probability as a value between 0 and 1. For example, a machine learning model can generate a probability that an individual has a fibrosis stage of F2 or greater as a value between 0 and 1. Youden's Index can be used to determine a threshold or cutoff for the probability. In this example, the machine learning model can predict a 0.7 chance that the individual has a fibrosis stage of F2 or greater. Youden's Index can determine that, for this particular machine learning model, all probabilities greater than or equal to 0.6 generate a positive prediction (e.g., a prediction that the individual does have liver fibrosis of F2 or greater). The threshold or cutoff generated by Youden's Index can be a percentage (e.g., a value between 0% and 100%) or a value between 0 and 1. For example, the cutoff generated by Youden's Index can be 90%.

Step 318 can include determining a fibrosis stage based on the liver score. The prediction service 218 can determine the fibrosis stage based on the liver score. For example, the liver score can include multiple scores, each indicating a fibrosis stage prediction. For example, a first score can predict if the individual has fibrosis stage of F2 or greater, a second score can predict if the individual has fibrosis stage of F3 or greater, and a third score can predict if the individual has fibrosis stage of F4. For example, the first score can predict that the individual has fibrosis stage of F2 or greater, the second score can predict that the individual has fibrosis of F3 or greater, and the third score can predict that the individual does not have fibrosis stage of F4. In this example, the fibrosis stage would be determined as F3 based on the liver scores. Step 318 can include determining a NAS based on the liver score. For example, the liver score can include multiple scores, each indicating a NAS prediction. For example, a first score can indicate if the individual has NAS of 4 or greater, a second score can indicate if the individual has NAS of 5 or greater, and a third score can indicate if the individual has NAS of 6 or greater. In this example, the first score can predict that the individual has a NAS of 4 or greater, the second score can predict that the individual has a NAS of 5 or greater, and the third score can predict that the individual does not have a NAS of 6 or greater. In this example, the NAS would be determined as 6 based on the liver scores.

Step 321 can include diagnosing a liver disease, including a fibrosis stage. The prediction service 218 can diagnose the liver disease. For example, if the fibrosis stage is F2 or greater and the NAS is 4 or greater, the individual can be diagnosed with NASH. For example, if the fibrosis stage is F4, the individual can be diagnosed with cirrhosis. The diagnoses can be determined based on the biology data 233 stored in the data store 221.

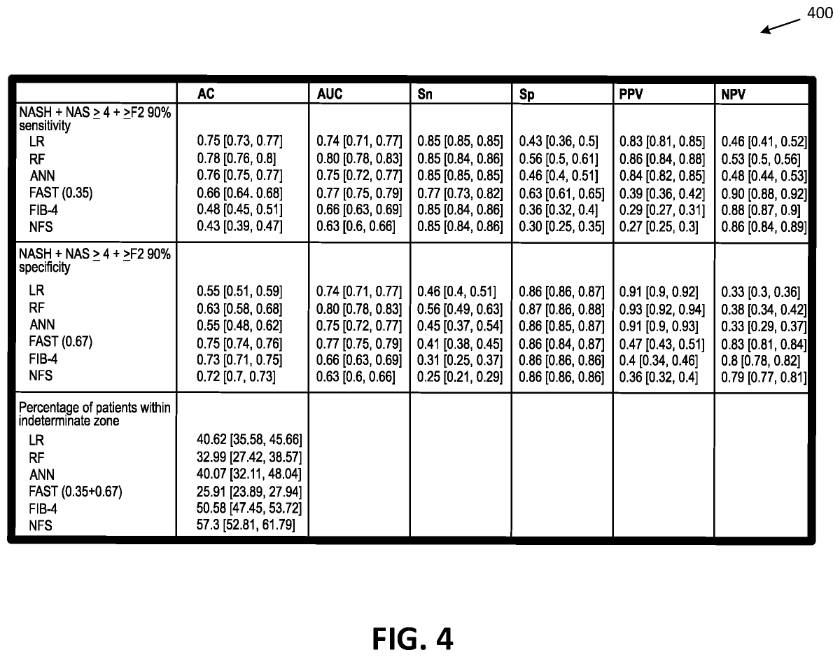

Referring now to FIG. 4, shown is an exemplary table 400 for according to various embodiments of the present disclosure. As will be understood, FIGS. 4-8 can illustrate the performance of various machine learning models against other diagnostic methods. The table 400 can show the performance of various different machine learning models compared to a FibroScan® test and a FIB-4 test. The FibroScan® test can be referred to as the FAST (Fibroscan-AST) test. LR refers to a logistic regression model, RF refers to a random forests model, and ANN refers to an artificial neural network model. The header of the rows (≥F2, ≥F3, ≥F4) refer to which fibrosis stage the test or model is configured to measure. “AC” refers to the accuracy, “AUC” refers to the area under the ROC curve, “Sn” refers to the sensitivity, “Sp” refers to the specificity, “PPV” refers to the positive predictive value, and “NPV” refers to the negative predictive value.

For ≥F2, RF had higher accuracy (which was defined as the overall proportion of patients correctly predicted), AUC, specificity, and PPV than FibroScan® (p<0.05). There was no statistically significant difference in sensitivity and NPV between RF and FibroScan® (p≥0.05). RF exhibited higher accuracy, AUC, sensitivity, and NPV than FIB-4 (p<0.05). There was no statistically significant difference in specificity and PPV between RF and FIB-4 (p≥0.05).

For ≥F3, RF displayed higher accuracy, AUC, specificity, and PPV than FibroScan® (p<0.05). There was no statistically significant difference in sensitivity and NPV between RF and FibroScan® (p≥0.05). RF displayed higher accuracy, AUC, sensitivity, and NPV than FIB-4 (p<0.05). However, RF displayed lower specificity than FIB-4 (p<0.05). There was no statistically significant difference in PPV between RF and FIB-4 (p≥0.05).

For F4, RF had higher accuracy, specificity, and PPV than FibroScan® (p<0.05). However, RF displayed lower sensitivity than FibroScan® (p<0.05). There was no statistically significant difference in AUC and NPV between RF and FibroScan® (p≥0.05). RF demonstrated higher accuracy, sensitivity, and NPV compared to FIB-4 (p<0.05). There was no statistically significant difference in AUC, specificity, and PPV between RF and FIB-4 (p≥0.05).

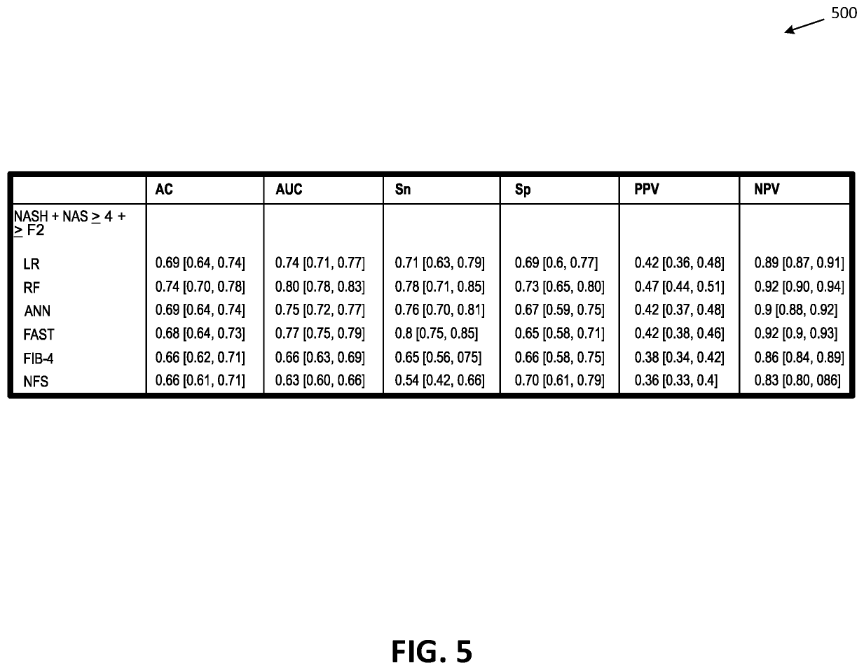

Referring now to FIG. 5, shown is an exemplary table 500 for according to various embodiments of the present disclosure. The table 500 can show the performance of various different machine learning models compared to a FibroScan® test, a FIB-4 test, and a FAST test. LR refers to a logistic regression model, RF refers to a random forests model, and ANN refers to an artificial neural network model. In table 500, each of the tests and models is configured to determine whether the patient has a fibrosis stage of at least F2, and a NAS of at least 4.

When using Youden's index to obtain the cutoffs, there was no statistically significant difference in accuracy, AUC, sensitivity, specificity, PPV, and NPV between RF and FAST (p≥0.05). However, the numbers overall were numerically higher. RF demonstrated higher AUC, PPV, and NPV than FIB-4 (p<0.05). There was no statistically significant difference in accuracy, sensitivity and specificity between RF and FIB-4 (p≥0.05). RF demonstrated higher AUC, sensitivity, PPV, and NPV than NFS (p<0.05). There was no statistically significant difference in accuracy and specificity between RF and NFS (p≥0.05).

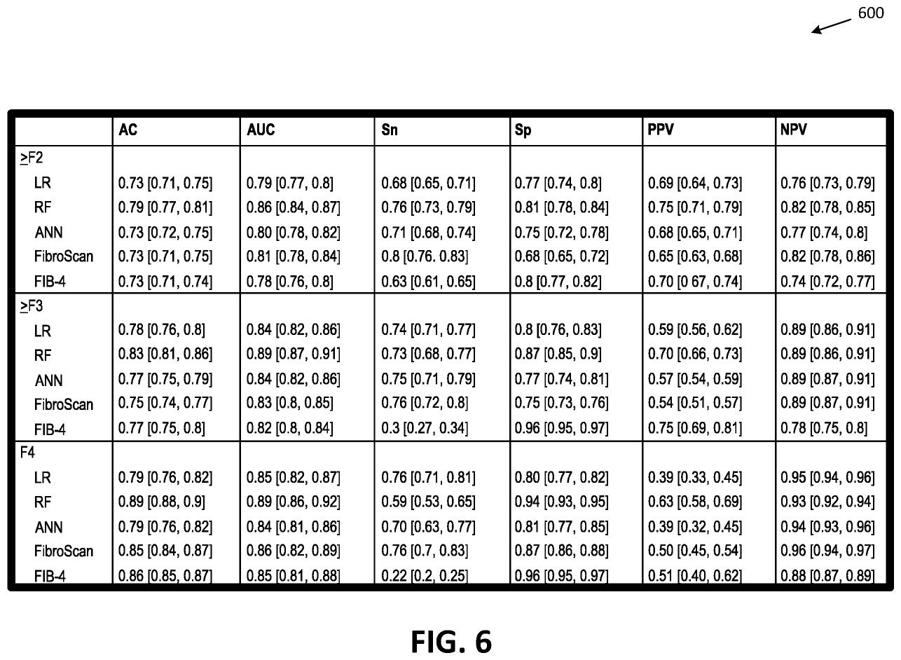

Referring now to FIG. 6, shown is an exemplary table 600 for according to various embodiments of the present disclosure. The table 600 can show the performance of same machine learning models compared to the same tests when a 90% sensitivity is used and when a 90% specificity is used. Table 600 also shows the percentage of patients within an indeterminate zone for each model and test. When using 90% sensitivity to obtain the cutoffs, RF demonstrated higher accuracy, sensitivity, and PPV than FAST (p<0.05). However, RF had lower NPV than FAST (p<0.05). There was no statistically significant difference in AUC and specificity between RF and FAST (p≥0.05). RF demonstrated higher accuracy, AUC, specificity, and PPV than FIB-4 or NFS (p<0.05). However, RF had lower NPV than FIB-4 or NFS (p<0.05). There was no statistically significant difference in sensitivity between RF and FIB-4 or NFS (p≥0.05).

When using 90% specificity to obtain the cutoffs, RF demonstrated higher sensitivity and PPV than FAST (p<0.05). However, RF exhibited lower accuracy and NPV than FAST (p<0.05). There was no statistically significant difference in specificity and AUC between RF and FAST (p≥0.05). RF demonstrated higher AUC, sensitivity, and PPV than FIB-4 or NFS (p<0.05). However, RF exhibited lower accuracy and NPV than FIB-4 or NFS (p<0.05). There was no statistically significant difference in specificity between RF and FIB-4 or NFS (p≥0.05).

Finally, for NASH+NAS≥4+≥F2, RF exhibited a lower percentage of patients within the indeterminate zone (between 90% sensitivity and 90% specificity) compared to FIB-4 and NFS (p<0.05). There was no statistically significant difference in the percentage of patients within the indeterminate zone between RF and FAST (p≥0.05).

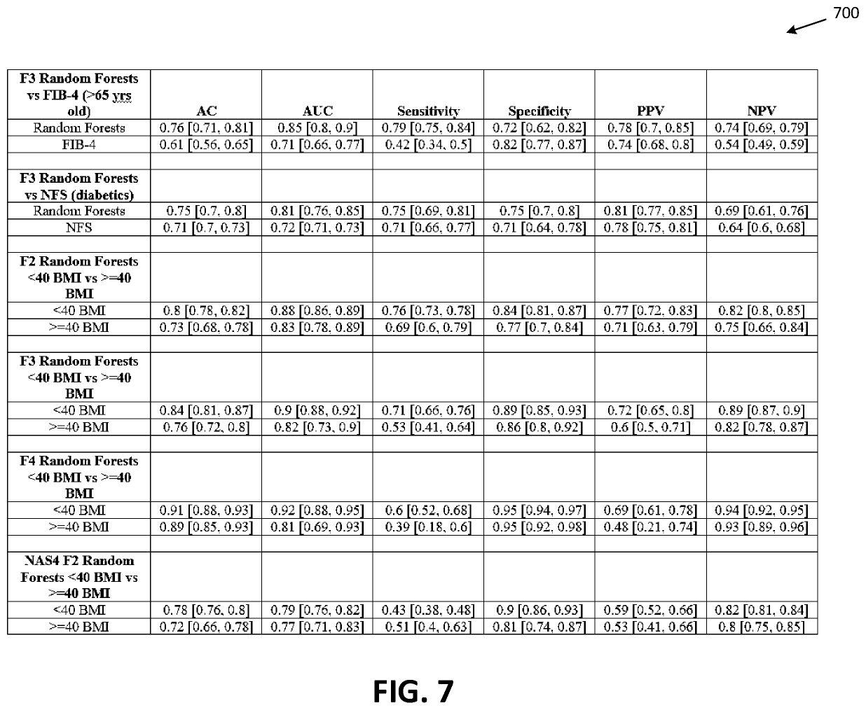

Referring now to FIG. 7, shown is an exemplary table 700 for according to various embodiments of the present disclosure. The table 700 can show the testing results from a variety of different models for different patient cohorts. When comparing RF to FIB-4 using a cutoff of ≥F3 for RF in a cohort containing only those age 65 years (yrs) or older, there was no statistically significant difference in specificity or PPV (p≥0.05). RF exhibited higher accuracy, AUC, sensitivity, and NPV compared to FIB-4 (p<0.05). When comparing RF to NFS using a cutoff of ≥F3 for RF in a cohort containing only diabetics, there was no statistically significant difference in accuracy, sensitivity, specificity, PPV, or NPV (p≥0.05). RF exhibited higher AUC (p<0.05).

When using a cutoff of ≥F2 for the RF model and comparing RF using a cohort containing those with BMI<40 kg/m 2 to itself using a cohort containing BMI≥40, there was no statistically significant difference in accuracy, AUC, sensitivity, specificity, PPV, or NPV (p≥0.05). When using a cutoff of ≥F3 for RF and doing the same comparison between RF and itself, there was no statistically significant difference in AUC, specificity, PPV, or NPV (p≥0.05). RF using the BMI<40 kg/m 2 cohort had higher accuracy and sensitivity than RF using the BMI≥40 kg/m 2 cohort (p<0.05). When using a cutoff of F4 and doing the same comparison, there was no statistically significant difference in accuracy, AUC, sensitivity, specificity, PPV, or NPV (p≥0.05). When using a cutoff of NASH with NAS≥4+>F2 and doing the same comparison, there was no statistically significant difference in accuracy, AUC, sensitivity, specificity, PPV, or NPV (p≥0.05).

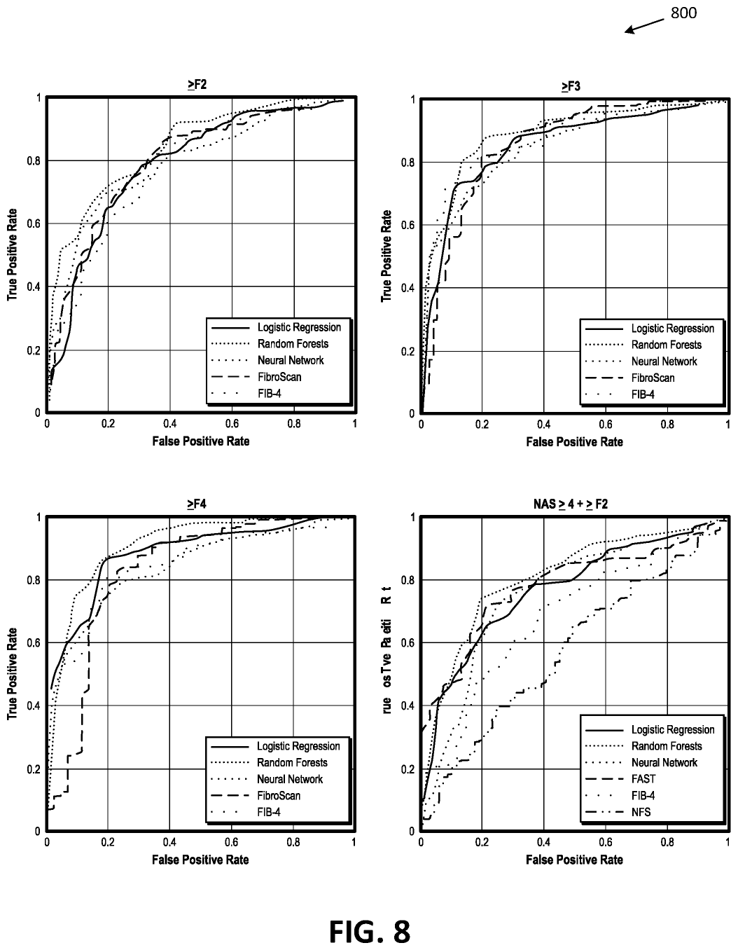

Referring now to FIG. 8, shown is an exemplary graphs 800 according to various embodiments of the present disclosure. The graphs 800 can show plots of the true positive rate versus the false positive rate for a logistic regression model, a random forests model, an artificial neural network model, a FibroScan® test, a FIB-4 test, and an NAFLD fibrosis score, in determining fibrosis stage≥F2, fibrosis stage≥F3, fibrosis stage≥F4, and a NAS≥4 and a fibrosis stage≥F2. As can be seen, the machine learning models compare favorably to existing tests.

Thus, among the machine learning (ML) models, RF almost always had higher AUC and accuracy than LR, ANN, or traditional NITs in identifying≥F2, ≥F3, F4, and NASH+NAS≥4+≥F2. Since AUC measures how well a model performs across a gradient of cutoffs, a higher AUC indicates that a model is better at distinguishing between the positive and negative classes. The accuracy is the percentage of correct predictions for test data, with higher accuracy indicating overall better performance. Therefore, these results suggest that RF demonstrated better discrimination in separating the positive and negative classes and performed better overall compared to LR and ANN. ML also performed better overall compared to other NITs (NFS, FIB-4, FibroScan®, FAST).

Additionally, for NASH with NAS≥4+≥F2, ML exhibited just as low or lower percentage of patients within the indeterminate zone (between 90% sensitivity and 90% specificity) compared to FAST, FIB-4 and NFS. This further proves that ML is just as capable of separating positive and negative classes.

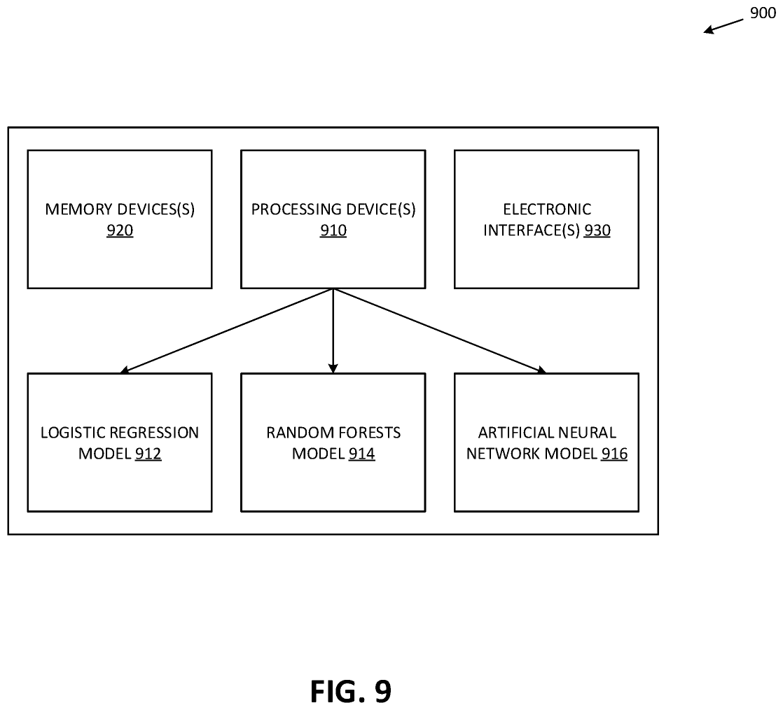

Referring now to FIG. 9, shown is an exemplary computing system 900 according to various embodiments of the present disclosure. The exemplary computing system 900 may be in addition or alternative to the exemplary networked environment 200. The computing system 900 can be used for implementing method 300 or any other methods or processes discussed herein. System 900 can include at least one processing device 910, at least one memory device 920, and at least one electronic interface 930. The processing device 910 can be configured to implement one or more machine learning models, which can include at least one logistic regression model 912, at least one random forests model 914, at least one artificial neural network model 916, other models, other machine learning models such as those described herein, or any combination thereof. The at least one memory device 920 can be configured to store any data needed for the execution of the machine learning models, including data associated with biological characteristic of the individual being analyzed. The at least one electronic interface 930 is configured to receive data from external sources, and can include a variety of different communication interfaces.

In some implementations, a non-transitory, machine/computer-readable medium has instructions stored thereon for implementing method 300 or any other methods or processes discussed herein. A machine processor is configured to executed the instructions in order to perform these methods or processes.

Aspects of the present disclosure can be implemented on a variety of types of processing devices, such as general purpose computer systems, microprocessors, digital signal processors, micro-controllers, application specific integrated circuits (ASICs), programmable logic devices (PLDs) field programmable logic devices (FPLDs), programmable gate arrays (PGAs), field programmable gate arrays (FPGAs), mobile devices such as mobile telephones, personal digital assistants (PDAs), or tablet computers, local servers, remote servers, wearable computers, or the like.

Memory storage devices of the one or more processing devices can include a machine-readable medium on which is stored one or more sets of instructions (e.g., software) embodying any one or more of the methodologies or functions described herein. The instructions can further be transmitted or received over a network via a network transmitter receiver. While the machine-readable medium can be a single medium, the term “machine-readable medium” should be taken to include a single medium or multiple media (e.g., a centralized or distributed database, and/or associated caches and servers) that store the one or more sets of instructions. The term “machine-readable medium” can also be taken to include any medium that is capable of storing, encoding, or carrying a set of instructions for execution by the machine and that cause the machine to perform any one or more of the methodologies of the various embodiments, or that is capable of storing, encoding, or carrying data structures utilized by or associated with such a set of instructions. The term “machine-readable medium” can accordingly be taken to include, but not be limited to, solid-state memories, optical media, and magnetic media. A variety of different types of memory storage devices, such as a random access memory (RAM) or a read only memory (ROM) in the system or a floppy disk, hard disk, CD ROM, DVD ROM, flash, or other computer readable medium that is read from and/or written to by a magnetic, optical, or other reading and/or writing system that is coupled to the processing device, can be used for the memory or memories.

From the foregoing, it will be understood that various aspects of the processes described herein are software processes that execute on computer systems that form parts of the system. Accordingly, it will be understood that various embodiments of the system described herein are generally implemented as specially-configured computers including various computer hardware components and, in many cases, significant additional features as compared to conventional or known computers, processes, or the like, as discussed in greater detail herein. Embodiments within the scope of the present disclosure also include computer-readable media for carrying or having computer-executable instructions or data structures stored thereon. Such computer-readable media can be any available media which can be accessed by a computer, or downloadable through communication networks. By way of example, and not limitation, such computer-readable media can comprise various forms of data storage devices or media such as RAM, ROM, flash memory, EEPROM, CD-ROM, DVD, or other optical disk storage, magnetic disk storage, solid state drives (SSDs) or other data storage devices, any type of removable nonvolatile memories such as secure digital (SD), flash memory, memory stick, etc., or any other medium which can be used to carry or store computer program code in the form of computer-executable instructions or data structures and which can be accessed by a general purpose computer, special purpose computer, specially-configured computer, mobile device, etc.

When information is transferred or provided over a network or another communications connection (either hardwired, wireless, or a combination of hardwired or wireless) to a computer, the computer properly views the connection as a computer-readable medium. Thus, any such connection is properly termed and considered a computer-readable medium. Combinations of the above should also be included within the scope of computer-readable media. Computer-executable instructions comprise, for example, instructions and data which cause a general purpose computer, special purpose computer, or special purpose processing device such as a mobile device processor to perform one specific function or a group of functions.

Those skilled in the art will understand the features and aspects of a suitable computing environment in which aspects of the disclosure may be implemented. Although not required, some of the embodiments of the claimed systems may be described in the context of computer-executable instructions, such as program modules or engines, as described earlier, being executed by computers in networked environments. Such program modules are often reflected and illustrated by flow charts, sequence diagrams, exemplary screen displays, and other techniques used by those skilled in the art to communicate how to make and use such computer program modules. Generally, program modules include routines, programs, functions, objects, components, data structures, application programming interface (API) calls to other computers whether local or remote, etc. that perform particular tasks or implement particular defined data types, within the computer. Computer-executable instructions, associated data structures and/or schemas, and program modules represent examples of the program code for executing steps of the methods disclosed herein. The particular sequence of such executable instructions or associated data structures represent examples of corresponding acts for implementing the functions described in such steps.

Those skilled in the art will also appreciate that the claimed and/or described systems and methods may be practiced in network computing environments with many types of computer system configurations, including personal computers (PCs), smartphones, tablets, hand-held devices, multi-processor systems, microprocessor-based or programmable consumer electronics, networked PCs, minicomputers, mainframe computers, and the like. Embodiments of the claimed system are practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing various aspects of the described operations, which is not illustrated, includes a computing device including a processing unit, a system memory, and a system bus that couples various system components including the system memory to the processing unit. The computer will typically include one or more data storage devices for reading data from and writing data to. The data storage devices provide nonvolatile storage of computer-executable instructions, data structures, program modules, and other data for the computer.

Computer program code that implements the functionality described herein typically comprises one or more program modules that may be stored on a data storage device. This program code, as is known to those skilled in the art, usually includes an operating system, one or more application programs, other program modules, and program data. A user may enter commands and information into the computer through keyboard, touch screen, pointing device, a script containing computer program code written in a scripting language or other input devices (not shown), such as a microphone, etc. These and other input devices are often connected to the processing unit through known electrical, optical, or wireless connections.

The computer that effects many aspects of the described processes will typically operate in a networked environment using logical connections to one or more remote computers or data sources, which are described further below. Remote computers may be another personal computer, a server, a router, a network PC, a peer device or other common network node, and typically include many or all of the elements described above relative to the main computer system in which the systems are embodied. The logical connections between computers include a local area network (LAN), a wide area network (WAN), virtual networks (WAN or LAN), and wireless LANs (WLAN) that are presented here by way of example and not limitation. Such networking environments are commonplace in office-wide or enterprise-wide computer networks, intranets, and the Internet.

When used in a LAN or WLAN networking environment, a computer system implementing aspects of the system is connected to the local network through a network interface or adapter. When used in a WAN or WLAN networking environment, the computer may include a modem, a wireless link, or other mechanisms for establishing communications over the wide area network, such as the Internet. In a networked environment, program modules depicted relative to the computer, or portions thereof, may be stored in a remote data storage device. It will be appreciated that the network connections described or shown are exemplary and other mechanisms of establishing communications over wide area networks or the Internet may be used.

While various aspects have been described in the context of a preferred embodiment, additional aspects, features, and methodologies of the claimed systems will be readily discernible from the description herein, by those of ordinary skill in the art. Many embodiments and adaptations of the disclosure and claimed systems other than those herein described, as well as many variations, modifications, and equivalent arrangements and methodologies, will be apparent from or reasonably suggested by the disclosure and the foregoing description thereof, without departing from the substance or scope of the claims. Furthermore, any sequence(s) and/or temporal order of steps of various processes described and claimed herein are those considered to be the best mode contemplated for carrying out the claimed systems. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed systems. In addition, some steps may be carried out simultaneously, contemporaneously, or in synchronization with other steps.

Aspects, features, and benefits of the claimed devices and methods for using the same will become apparent from the information disclosed in the exhibits and the other applications as incorporated by reference. Variations and modifications to the disclosed systems and methods may be effected without departing from the spirit and scope of the novel concepts of the disclosure.

It will, nevertheless, be understood that no limitation of the scope of the disclosure is intended by the information disclosed in the exhibits or the applications incorporated by reference; any alterations and further modifications of the described or illustrated embodiments, and any further applications of the principles of the disclosure as illustrated therein are contemplated as would normally occur to one skilled in the art to which the disclosure relates.

The foregoing description of the exemplary embodiments has been presented only for the purposes of illustration and description and is not intended to be exhaustive or to limit the devices and methods for using the same to the precise forms disclosed. Many modifications and variations are possible in light of the above teaching.

The embodiments were chosen and described in order to explain the principles of the devices and methods for using the same and their practical application so as to enable others skilled in the art to utilize the devices and methods for using the same and various embodiments and with various modifications as are suited to the particular use contemplated. Alternative embodiments will become apparent to those skilled in the art to which the present devices and methods for using the same pertain without departing from their spirit and scope. Accordingly, the scope of the present devices and methods for using the same is defined by the appended claims rather than the foregoing description and the exemplary embodiments described therein.

Clause 1. A method, comprising: receiving, via one of one or more computing devices, a plurality of sets of historical biological data corresponding to a plurality of individuals, wherein each of the plurality of sets of historical biological data comprise a respective diagnosis of at least one disease associated with a respective liver; generating, via one of the one or more computing devices, at least one machine learning model predictive of the at least one disease; training, via one of the one or more computing devices, the at least one machine learning model using the plurality of sets of historical biological data; receiving, via one of the one or more computing devices, data associated with at least one biological characteristic of a particular individual; and generating, via one of the one or more computing devices, at least one liver score predictive of the at least one disease in the particular individual by applying the at least one machine learning model to the data associated with the at least one biological characteristic of the particular individual.

Clause 2. The method of clause 1 or any other clause herein, wherein applying the at least one machine learning model comprises: generating at least one formula from the at least one machine learning model; and applying the at least one formula to the data associated with the at least one biological characteristic of the particular individual.

Clause 3. The method of clause 1 or any other clause herein, wherein the historical biological data comprises a plurality of biological features and the at least one biological characteristic corresponds to the plurality of biological features.

Clause 4. The method of clause 3 or any other clause herein, wherein the plurality of biological features comprises: body mass index (BMI), alkaline phosphatase, total bilirubin, alanine transaminase (ALT), aspartate aminotransferase (AST), albumin, white blood count (WBC), platelet count, hemoglobin A1C, total cholesterol, low-density lipoprotein (LDL) cholesterol, high-density lipoprotein (HDL), triglycerides, type 2 diabetes status, and hypertension status.

Clause 5. The method of clause 1 or any other clause herein, wherein the at least one machine learning model comprises a plurality of machine learning models individually predictive of a respective fibrosis stage of a liver and the at least one liver score comprises a plurality of liver scores each predictive of a respective fibrosis stage of a liver.

Clause 6. The method of clause 1 or any other clause herein, wherein the at least one machine learning model comprises a plurality of machine learning models individually predictive of a respective non-alcoholic fatty liver (NAFLD) disease activity score (NAS) of a liver and the at least one liver score comprises a plurality of liver scores each predictive of a NAS of a liver.

Clause 7. The method of clause 1 or any other clause herein, further comprising: determining, via one of the one or more computing devices, a particular fibrosis stage of a plurality of stages based on the at least one liver score; and diagnosing, via one of the one or more computing devices, the particular fibrosis stage for the particular individual.

Clause 8. A system, comprising: a data store; and at least one computing device in communication with the data store, wherein the at least one computing device is configured to: receive a plurality of sets of historical biological data corresponding to a plurality of individuals, wherein the plurality of sets of historical biological data comprises a plurality of biological features and each of the plurality of sets of historical biological data comprise a respective diagnosis of at least one disease associated with a respective liver; generate at least one machine learning model predictive of the at least one disease; train the at least one machine learning model using the plurality of sets of historical biological data; receive data associated with at least one biological characteristic of a particular individual; and generate at least one liver score predictive of the at least one disease in the particular individual by applying the at least one machine learning model to the data associated with the at least one biological characteristic of the particular individual.

Clause 9. The system of clause 8 or any other clause herein, further comprising an electronic interface configured to receive the data associated with the at least one biological characteristic of the particular individual.

Clause 10. The system of clause 8 or any other clause herein, wherein the at least one computing device is further configured to receive a plurality of second sets of historical biological data corresponding to a plurality of second individuals, wherein the plurality of second sets of historical biological data comprises the plurality of biological features and each of the plurality of second sets of historical biological data comprise a respective indication of non-diagnosis of at least one disease associated with a respective liver.

Clause 11. The system of clause 8 or any other clause herein, wherein the at least one liver score comprises a first score that is predictive of whether the particular individual has a fibrosis stage at or above F2, a second score that is predictive of whether the particular individual has a fibrosis stage at or above F3, and a third score that is predictive of whether the particular individual has a fibrosis stage of F4.

Clause 12. The system of clause 8 or any other clause herein, wherein the at least one machine learning model comprises a decision tree model using a plurality of random subsets of the plurality of biological features.

Clause 13. The system of clause 12 or any other clause herein, wherein the at least one computing device is further configured to: generate a prediction for each tree in the decision tree model; and generate an output of a highest ranking prediction across trees in the decision tree model.

Clause 14. The system of clause 12 or any other clause herein, wherein the decision tree model comprises at least 30 decisions trees and excludes a maximum tree depth.

Clause 15. The system of clause 8 or any other clause herein, wherein the plurality of biological features comprise at least one of: body mass index (BMI), alkaline phosphatase, total bilirubin, alanine transaminase (ALT), aspartate aminotransferase (AST), albumin, white blood count (WBC), platelet count, hemoglobin A1C, total cholesterol, low-density lipoprotein (LDL) cholesterol, high-density lipoprotein (HDL), triglycerides, type 2 diabetes status, or hypertension status.

Clause 16. A non-transitory computer-readable medium embodying a program that, when executed by at least one computing device, causes the at least one computing device to: receive a plurality of sets of historical biological data corresponding to a plurality of individuals, wherein each of the plurality of sets of historical biological data comprise a respective diagnosis of at least one disease associated with a respective liver; generate at least one machine learning model predictive of the at least one disease; train the at least one machine learning model using the plurality of sets of historical biological data; receive data associated with at least one biological characteristic of a particular individual; and generate at least one liver score predictive of the at least one disease in the particular individual by applying the at least one machine learning model to the data associated with the at least one biological characteristic of the particular individual.

Clause 17. The non-transitory computer-readable medium of clause 16 or any other clause herein, wherein the historical biological data comprises a plurality of biological features and the plurality of biological features are selected from: body mass index (BMI), alkaline phosphatase, total bilirubin, alanine transaminase (ALT), aspartate aminotransferase (AST), albumin, white blood count (WBC), platelet count, hemoglobin A1C, total cholesterol, low-density lipoprotein (LDL) cholesterol, high-density lipoprotein (HDL), triglycerides, type 2 diabetes status, and hypertension status.

Clause 18. The non-transitory computer-readable medium of clause 16 or any other clause herein, wherein the at least one machine learning model comprises at least one of: a logistic regression model, a random forests model, or an artificial neural network.

Clause 19. The non-transitory computer-readable medium of clause 16 or any other clause herein, wherein the at least one machine learning model comprises at least two hidden layers.

Clause 20. The non-transitory computer-readable medium of clause 16 or any other clause herein, wherein one of the at least one machine learning model, when executed by the at least one computing device, is configured to determine an indication of whether a fibrosis stage of a liver of the particular individual is greater than or equal to F2 and a NAS of the liver of the particular individual is greater than or equal to 4.

Clause 21. The non-transitory computer-readable medium of clause 16 or any other clause herein, wherein the program further causes the at least one computing device to determine a diagnosis of a stage of a liver disease based on the at least one liver score and a 90% cutoff generated using Youden's index.

These and other aspects, features, and benefits of the claims will become apparent from the detailed written description of the aforementioned aspects taken in conjunction with the accompanying drawings, although variations and modifications thereto may be effected without departing from the spirit and scope of the novel concepts of the disclosure.

What is claimed is:

1. A method, comprising:
receiving, via one of one or more computing devices, a plurality of sets of historical biological data corresponding to a plurality of individuals, wherein each of the plurality of sets of historical biological data comprise a respective diagnosis of at least one disease associated with a respective liver;
generating, via one of the one or more computing devices, at least one machine learning model predictive of the at least one disease;
training, via one of the one or more computing devices, the at least one machine learning model using the plurality of sets of historical biological data;
receiving, via one of the one or more computing devices, data associated with at least one biological characteristic of a particular individual; and
generating, via one of the one or more computing devices, at least one liver score predictive of the at least one disease in the particular individual by applying the at least one machine learning model to the data associated with the at least one biological characteristic of the particular individual.

2. The method of claim 1, wherein applying the at least one machine learning model comprises:
generating at least one formula from the at least one machine learning model; and
applying the at least one formula to the data associated with the at least one biological characteristic of the particular individual.

3. The method of claim 1, wherein the historical biological data comprises a plurality of biological features and the at least one biological characteristic corresponds to the plurality of biological features.

4. The method of claim 3, wherein the plurality of biological features comprises: body mass index (BMI), alkaline phosphatase, total bilirubin, alanine transaminase (ALT), aspartate aminotransferase (AST), albumin, white blood count (WBC), platelet count, hemoglobin A1C, total cholesterol, low-density lipoprotein (LDL) cholesterol, high-density lipoprotein (HDL), triglycerides, type 2 diabetes status, and hypertension status.

5. The method of claim 1, wherein the at least one machine learning model comprises a plurality of machine learning models individually predictive of a respective fibrosis stage of a liver and the at least one liver score comprises a plurality of liver scores each predictive of a respective fibrosis stage of a liver.

6. The method of claim 1, wherein the at least one machine learning model comprises a plurality of machine learning models individually predictive of a respective non-alcoholic fatty liver (NAFLD) disease activity score (NAS) of a liver and the at least one liver score comprises a plurality of liver scores each predictive of a NAS of a liver.

7. The method of claim 1, further comprising:
determining, via one of the one or more computing devices, a particular fibrosis stage of a plurality of stages based on the at least one liver score; and
diagnosing, via one of the one or more computing devices, the particular fibrosis stage for the particular individual.

8. A system, comprising:
a data store; and
at least one computing device in communication with the data store, wherein the at least one computing device is configured to:
receive a plurality of sets of historical biological data corresponding to a plurality of individuals, wherein the plurality of sets of historical biological data comprises a plurality of biological features and each of the plurality of sets of historical biological data comprise a respective diagnosis of at least one disease associated with a respective liver;
generate at least one machine learning model predictive of the at least one disease;
train the at least one machine learning model using the plurality of sets of historical biological data;
receive data associated with at least one biological characteristic of a particular individual; and
generate at least one liver score predictive of the at least one disease in the particular individual by applying the at least one machine learning model to the data associated with the at least one biological characteristic of the particular individual.

9. The system of claim 8, further comprising an electronic interface configured to receive the data associated with the at least one biological characteristic of the particular individual.

10. The system of claim 8, wherein the at least one computing device is further configured to receive a plurality of second sets of historical biological data corresponding to a plurality of second individuals, wherein the plurality of second sets of historical biological data comprises the plurality of biological features and each of the plurality of second sets of historical biological data comprise a respective indication of non-diagnosis of at least one disease associated with a respective liver.

11. The system of claim 8, wherein the at least one liver score comprises a first score that is predictive of whether the particular individual has a fibrosis stage at or above F2, a second score that is predictive of whether the particular individual has a fibrosis stage at or above F3, and a third score that is predictive of whether the particular individual has a fibrosis stage of F4.

12. The system of claim 8, wherein the at least one machine learning model comprises a decision tree model using a plurality of random subsets of the plurality of biological features.

13. The system of claim 12, wherein the at least one computing device is further configured to:
generate a prediction for each tree in the decision tree model; and
generate an output of a highest ranking prediction across trees in the decision tree model.

14. The system of claim 12, wherein the decision tree model comprises at least 30 decisions trees and excludes a maximum tree depth.

15. The system of claim 8, wherein the plurality of biological features comprise at least one of: body mass index (BMI), alkaline phosphatase, total bilirubin, alanine transaminase (ALT), aspartate aminotransferase (AST), albumin, white blood count (WBC), platelet count, hemoglobin A1C, total cholesterol, low-density lipoprotein (LDL) cholesterol, high-density lipoprotein (HDL), triglycerides, type 2 diabetes status, or hypertension status.

16. A non-transitory computer-readable medium embodying a program that, when executed by at least one computing device, causes the at least one computing device to:
receive a plurality of sets of historical biological data corresponding to a plurality of individuals, wherein each of the plurality of sets of historical biological data comprise a respective diagnosis of at least one disease associated with a respective liver;
generate at least one machine learning model predictive of the at least one disease;
train the at least one machine learning model using the plurality of sets of historical biological data;
receive data associated with at least one biological characteristic of a particular individual; and
generate at least one liver score predictive of the at least one disease in the particular individual by applying the at least one machine learning model to the data associated with the at least one biological characteristic of the particular individual.

17. The non-transitory computer-readable medium of claim 16, wherein the historical biological data comprises a plurality of biological features and the plurality of biological features are selected from: body mass index (BMI), alkaline phosphatase, total bilirubin, alanine transaminase (ALT), aspartate aminotransferase (AST), albumin, white blood count (WBC), platelet count, hemoglobin A1C, total cholesterol, low-density lipoprotein (LDL) cholesterol, high-density lipoprotein (HDL), triglycerides, type 2 diabetes status, and hypertension status.

18. The non-transitory computer-readable medium of claim 16, wherein the at least one machine learning model comprises at least one of: a logistic regression model, a random forests model, or an artificial neural network.

19. The non-transitory computer-readable medium of claim 16, wherein the at least one machine learning model comprises at least two hidden layers.

20. The non-transitory computer-readable medium of claim 16, wherein one of the at least one machine learning model, when executed by the at least one computing device, is configured to determine an indication of whether a fibrosis stage of a liver of the particular individual is greater than or equal to F2 and a NAS of the liver of the particular individual is greater than or equal to 4.

21. The non-transitory computer-readable medium of claim 16, wherein the program further causes the at least one computing device to determine a diagnosis of a stage of a liver disease based on the at least one liver score and a 90% cutoff generated using Youden's index.
